(19) United States
(12) Patent Application Publication
(10) Pub. No.: US 2016/0228209 A1
(43) Pub. Date: Aug. 11, 2016

(54) ACTIVE ORTHOSIS FOR SURGEONS

(71) Applicants: Mauricio PLAZA TORRES, Bogota (CO); William APERADOR CHAPARRO, Bogota (CO); Oscar AVILES SANCHEZ, Bogota (CO); UNIVERSIDAD MILITAR NUEVA GRANADA, Bogotá (CO)

(72) Inventors: Mauricio PLAZA TORRES, Bogota (CO); William APERADOR CHAPARRO, Bogota (CO); Oscar AVILES SANCHEZ, Bogota (CO)

(21) Appl. No.: 14/916,898

(22) PCT Filed: Feb. 27, 2014
(86) PCT No.: PCT/IB2014/059305

§ 371 Date: Mar. 4, 2016

(30) Foreign Application Priority Data
Feb. 28, 2013 (CO) 13-040936

(51) Int. Cl. A61B 90/60

(52) U.S. Cl. CPC A61B 90/60

(57) ABSTRACT

An orthosis according to definition is an external support or other device applied to the body to change the functional or structural aspects of the neuromuscular skeletal system.

The device which has been designed and described in detail above is intended to reduce the loads which surgeons have to support on their shoulders and legs during long periods of time during which they are engaged in surgical operations and therefore to reduce the risk that they will suffer lesions such as disc hernias, varicose veins, and pain, among others, through the use of mechatronic systems which facilitate their use and allow greater freedom of movement.

### TECHNICAL SECTOR

This invention relates to engineering applied to medicine (bioengineering) and relates to a device for use in operating theaters to reduce the risks of shoulder lesions in surgeons and to release pressures on the lower limbs, through supports reducing the loads which their musculoskeletal systems have to support during long-term surgery.

The said active orthosis device provides support to the pelvic region, reduces loads supported by the lower shoulder, does not inconvenience the surgeon during surgery and avoids damage to the legs such as varicose veins through reducing the loads which the legs have to support.

### STATE OF THE ART

At the present time many exoskeletons are being produced with a view to overcoming human limitations or in some cases to make good deficits, and are used in many fields, from the military to rehabilitation. Most exoskeletons are designed for the lower limbs, because it is this part of the body that is responsible for supporting most of the body's weight and for locomotion, and is also the most vulnerable to lesions.

There are devices which have provided great advances in this type of application thanks to their construction which requires robust design and advanced techniques of construction, one of the most well-known in the military field being the “Berkeley Lower Extremity Exoskeleton (BLEEX)” which developed fundamental technologies associated with the design and control of an electrically autonomous exoskeleton for the lower limbs, increasing strength and performance during locomotion¹. On the other hand there is the cyberdyne HAL, one of the most advanced exoskeletons, designed from the outset to assist the elderly and disabled persons². ¹ Kazerooni H. Exoskeletons for Human Power Augmentation. IEEE/RSJ International Conference on Intelligent Robots and Systems, August 2005.² Kawamura Y., “Study on Exoskeleton Power Assist HAL for walking Aid using EMG.” Nippon Robotto Gakkai Gakujutsu Koenkai Yokoshu Vol 18, 2000.

As far as exoskeletons for medical applications are concerned, various attempts have been made to obtain a device which reduces risks of shoulder lesions in surgeons. Many patent documents have been generated in the work of developing this device, including patent US 20080045374 which describes a rehabilitation device (AKROD) which is centered on the knee joint and has developed a very innovative design for the rehabilitation of patients with cerebrovascular accidents suffering from hyperextension of the knee in the stage of supporting walking and reduced flexion of the knee during the balancing stage.

Another document relating to a small gluteo-lumbar support device for use by surgeons (dental or medical) or auxiliary staff is patent ES 2129284. The device in this document comprises a seat and a back which can be adjusted for height through a threaded spindle and is supported on a plurality of feet or supports, at least five, with a system of rotation on ball bearings. This device is especially designed for use by dentists and their auxiliary staff as it enables them to maintain the position of maximum equilibrium between all the body segments involved in the working posture and thus avoid problems relating to long hours of working in the same position. However this device merely corresponds to a seat with an ergonomic support, because it does not have any type of motor or electronic control to assist support of the parts or the surgeon's body and thus avoid the problems associated with shoulders, lower limbs, etc.

To supplement the existing information in the state of the art it is necessary to include document US 2008249438 which discloses a training device and a method for use of the same to provide assistance to the movement of a patient's legs, in which the device comprises a supporting structure and one or more leg orthoses attached to the supporting structure, in which each leg orthosis comprises a thigh member connected to the supporting structure at a hip joint, and a leg member attached to the thigh member at a knee joint. Thus the two limbs have their respective connectors to secure them to the corresponding parts of the patient's body. The said equipment is to assist patients in their treatment for walking or other similar activity, and not to assist a surgeon when engaged in surgery. Likewise the said equipment does not have any type of electronic system or motors to assist movement.

To reduce shoulder lesions in surgeons during surgery high stools or seats inclining the pelvis have been used³, but although the said solutions are static they do not adjust to the surgeon's need for movement and cannot be used except in a few types of surgery with limitations as regards position and degrees of freedom. ³ Esser Adam C., MD, James G. Koshy, Ph.D, Henry W. Randle, MD, Ph.D. “Ergonomics in Office-Based Surgery: A Survey-Guided Observational Study”. American Society for Dermatologic Surgery, Inc. November 2007

It is clear that in the state of the art there are no dynamic devices with the ability to provide support for the pelvic region and reduce the loads supported by the lower shoulder, without inconveniencing surgeons during surgery.

Considering the above information it is obvious that there would be a need in the state of the art for devices reducing the risks of shoulder lesions in surgeons, which are dynamic in nature.

### DESCRIPTION OF THE FIGURES

The invention may be better understood by means of the figures which each show the components making up the active orthosis according to the invention. In addition to this the figures show the reference numbers assigned to the components making up the said device.

FIG. 1. Conceptual design of the active orthosis. a) Front view, b) side view, c) perspective view.

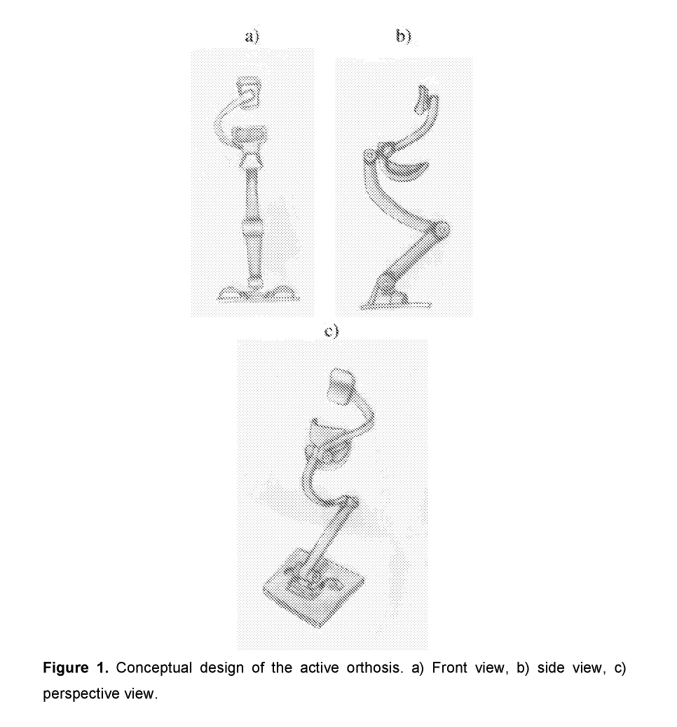

FIG. 2. Parts of the active orthosis according to the invention.

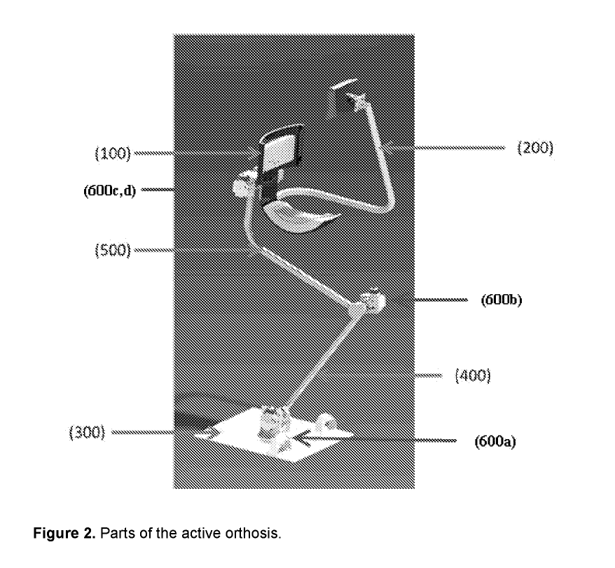

FIG. 3. View of the assembly with actuators and immobilizing system. b) Detail of the system for locking the articulations at the lower junction between the legs and base.

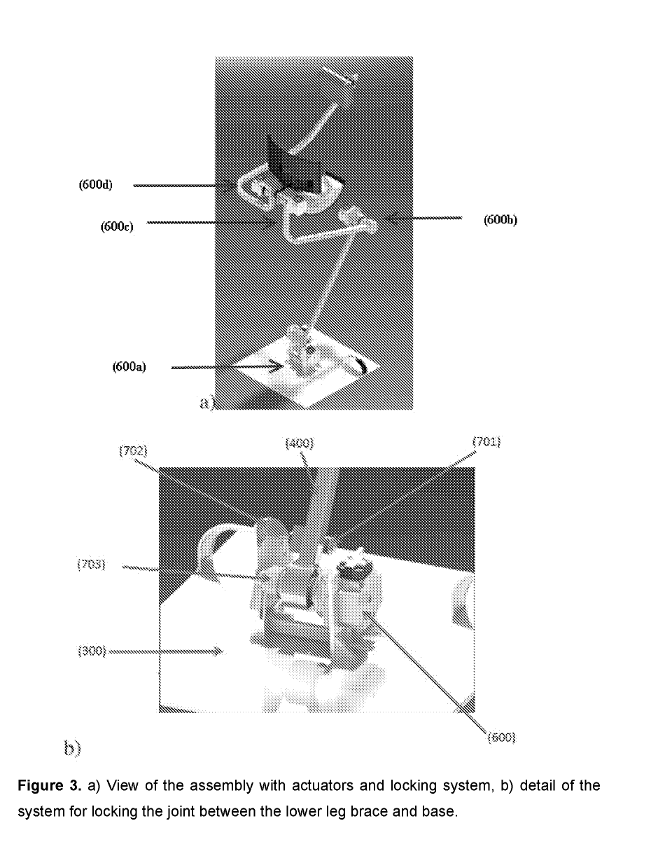

FIG. 4. Exploded view of each of the components of the device according to the invention.

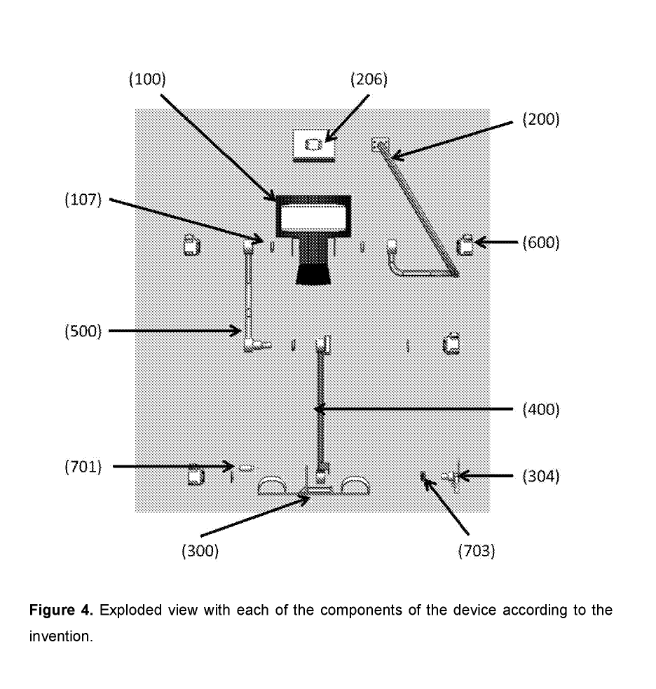

FIG. 5. Views of the base of the device.

FIG. 6. Views of the supporting shaft for the lower articulation.

FIG. 7. Views of the lower brace for the legs.

FIG. 8. Views of the upper brace for the legs.

FIG. 9. Views of the support for the pelvis.

FIG. 10. Views of the support for the chest.

FIG. 11. Examples of user positions which can be programmed in the device according to the invention.

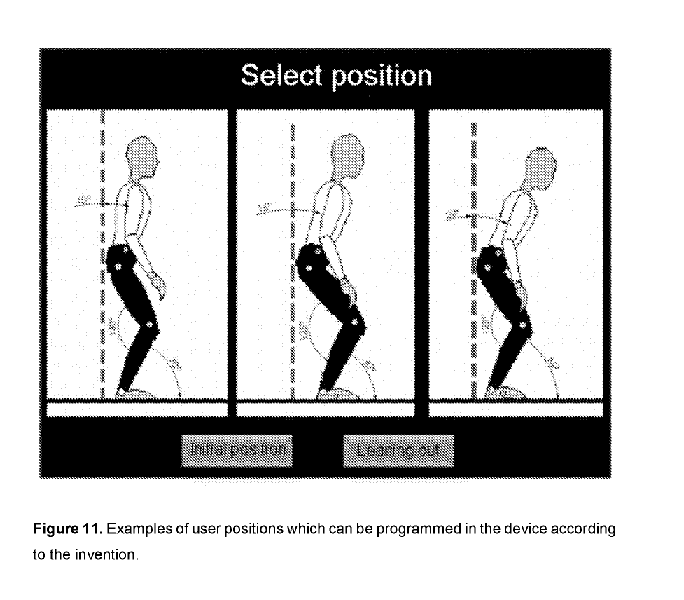

FIG. 12. Diagram of control of the device.

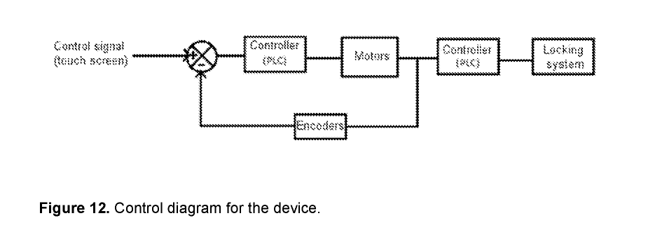

### DETAILED DESCRIPTION OF THE INVENTION

This invention relates to a device for use in operating theaters to reduce loads on surgeon's joints during long-term surgery. This device is of an active nature, which means that it can be dynamically adjusted with predetermined positions at times during surgery through control using electronic circuits.

The device provides support for the pelvic region, reduces the loads supported by the lower shoulder, and can be adjusted to programmed positions to avoid occupational health problems such as varicose veins, as well as many other problems in those professionals who remain in a standing position for long periods. The said device comprises a mechanical structure which is capable of being locked and an electronic control structure. Likewise it has a touch screen which can be used to control positions in accordance with the surgeon's posture and needs.

The device (1) according to this invention has a support for the pelvis (100) which makes it possible to incline it in accordance with surgeons' needs, thus reducing the forces exerted on the lower joints. The device also includes a chest support (200), which reduces the loads supported by the shoulder, transmitting them to the device. The structure supports the surgeon's weight and adjusts to programmed positions which are most frequent in different types of surgery.

Mechanical Structure

The structure of the device (1) has 5 fundamental parts, which are the base (300), the lower leg brace (400), the upper leg brace (500), the pelvis support (100) and the chest support (200).

The device (1) according to this invention may be made of stainless steel, given that it will be used within an operating theater and it is necessary that it should be easy to clean and should not suffer damage by contamination with fluids such as blood. This material can also easily be sterilized, has mechanical resistance to weight and movement conditions.

The device according to the invention additionally comprises four identical motors (600a, 600b, 600c, 600d) mounted in the various articulations of the device (2, 3, 4 and 5) to achieve the positions which are needed when it is in operation. Articulation (2) corresponds to the lower articulation which lies between the base (300) and the lower leg brace (400). Articulation (3) corresponds to the lower middle articulation which lies between the lower leg brace (400) and the upper leg brace (500). Articulation (4) corresponds to the upper middle articulation which lies between the upper leg brace (500) and the pelvis support (100). Articulation (5) corresponds to the upper articulation which lies between the upper leg brace (500) and the chest support (200).

The motors (600) must provide a torque of between 11 Nm and 18 Nm and an angular velocity of between 60 rad/min and 70 rad/min, which are ideal for reducing the loads which have to support the surgeon's weight. Motors (600) comprise an incorporated coding unit (601) which has a resolution of between 2000 pulses and 2500 pulses per revolution. The said coding unit (601) is used to control the position of the motor (600).

The structure of the device (1) is designed with a system of springs (6) which reduce the loads that have to be transmitted by the motors (600) and a locking system (700) in the lower articulation (2) which helps to support the various positions programmed for the device.

This locking system (700) comprises a solenoid (701) and a pin guide (702) in such a way that the solenoid (701) keeps the articulation locked in one position until it is decided to change to another, at which time the pin retracts and the motor (600a) is operated simultaneously, moving the device to the new position required, where solenoid (701) is activated again and locks articulation (2) in the new position.

A detailed description of the components of the device (1) is illustrated below:

Base Component (300):

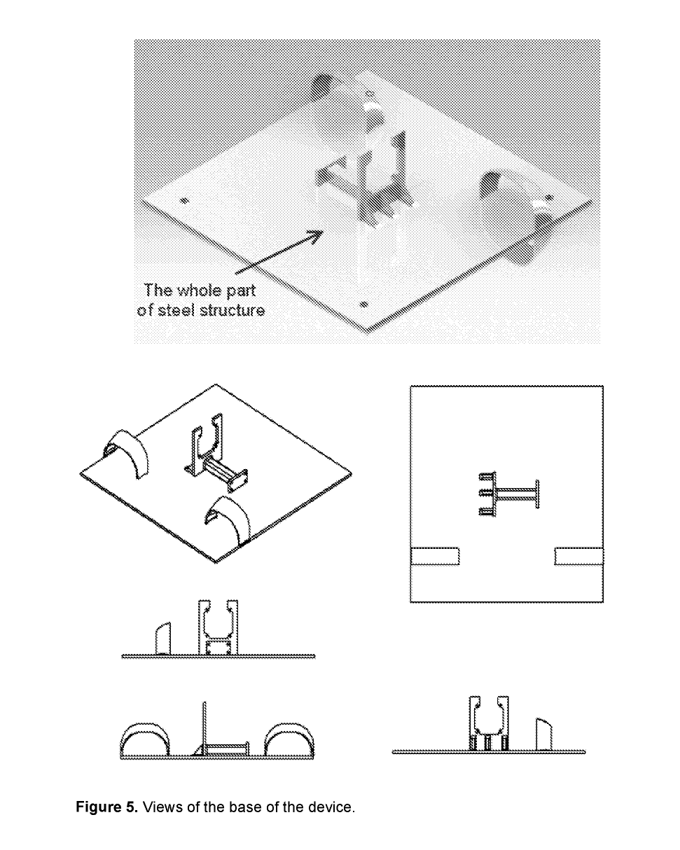

FIG. 5 shows the base (300) of device (1) intended to support the entire structure, as a result of which the said base comprises holes (301) for anchoring to the floor by means of bolts.

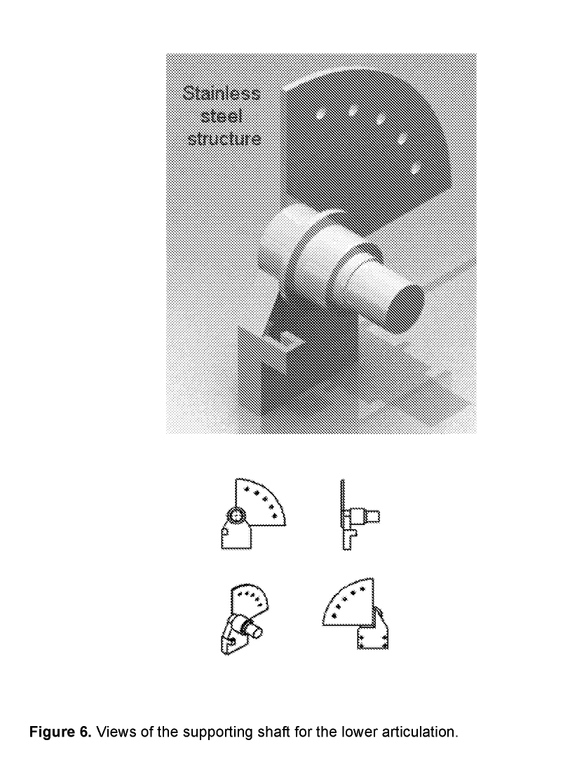

Base (300) corresponds to a flat sheet which has foot supports (302), so that the user of the device can conveniently position himself upon it; it likewise has a main support (303) on which a motor (600a) can be mounted, a shaft supporting lower articulation (304) and lower leg brace (400) on base (300). The said support shaft for the lower articulation (304) is as shown in FIG. 6 designed with a view to fitting lower leg brace (400) to base (300) and motor (600a).

This shaft supporting lower articulation (304) has a perforated circular support (702) for locking system (700), which the shaft of the solenoid (701) can enter in the different positions programmed for the device. Likewise the said support shaft for the lower articulation (304) comprises a mount (305) used to house a torsion spring (703) and limit this movement around the shaft.

Base (300) of device (1) and the components thereof are preferably made of stainless steel.

Lower Leg Brace (400):

As its name states, this component (400) of the structure is the one responsible for joining articulations (2 and 3) of the structure, which correspond to movements of the user's knee and ankle.

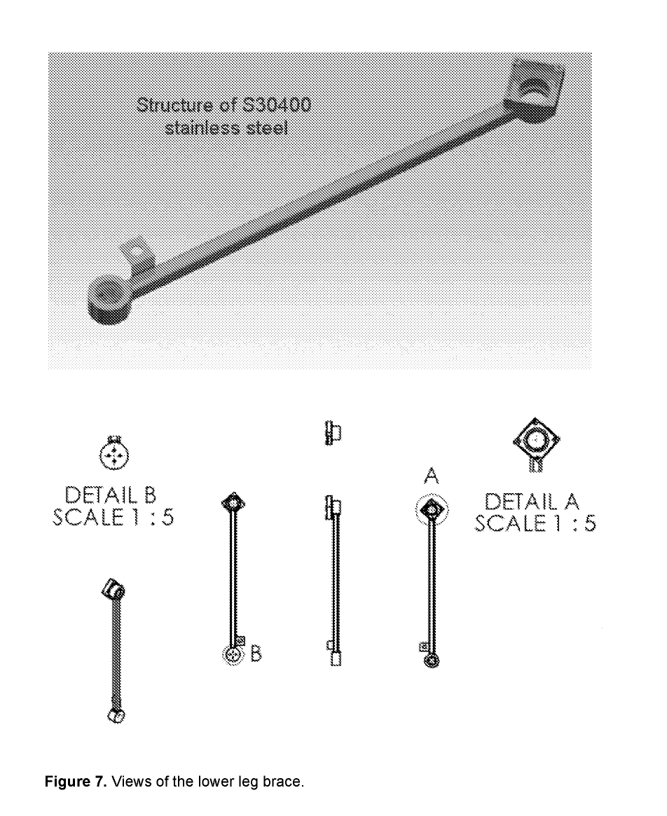

Lower leg brace (400) comprises a square structural component (401) and two shafts (402 and 403) each of which are located at the extremities of structural component (401). At the lower extremity there is a shaft (402) for connection to a radial ball race (404) supported on the supporting shaft for the lower articulation (304) and is also designed to be connected to the shaft of the lower motor (600a). At the upper extremity there is a hollow shaft (403) which can be used to house the body of the motor (600b) and allow the lower shaft (502) of the upper leg brace (500) to be connected to the motor (600b). Lower leg brace (400) shown in FIG. 7 also comprises a support (404) to anchor solenoid (701) of locking system (700) to the structure of lower leg brace (400).

Lower brace (400) of device (1) and its components are preferably made of stainless steel.

Upper Leg Brace.

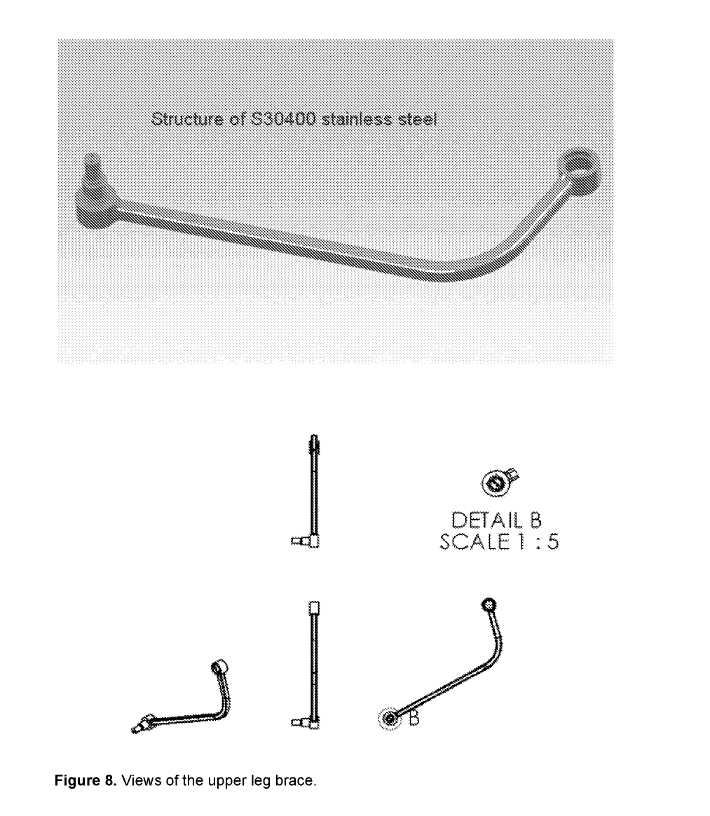

The upper leg brace (500) illustrated in FIG. 8 is the one responsible for making the connection between pelvis support (100) and lower leg brace (400). Said upper brace (500) comprises a square structural element (501) with an angle (504) of between 30° and 35°, and two shafts machined at the ends (502 and 503). The said angle (504) permits the pelvis of the user of device (1) to rotate around the shaft at the upper extremity (503) in such a way that the upper brace (500) does not impact against pelvis support (100).

The machined shaft at the lower extremity (502) has two shafts of different diameters so that the wider fits within ball race (404) and allows lower leg brace (400) to be housed while the smaller passes to allow upper leg brace (500) to house motor shaft (600b) housed in hollow shaft (403) at the upper extremity of lower leg brace (400) so that there is relative rotation between lower brace (400) and upper brace (500) which allows the knee of the user of device (1) to flex and extend.

Upper brace (500) of device (1) and its components are preferably made of stainless steel.

Pelvis Support (100):

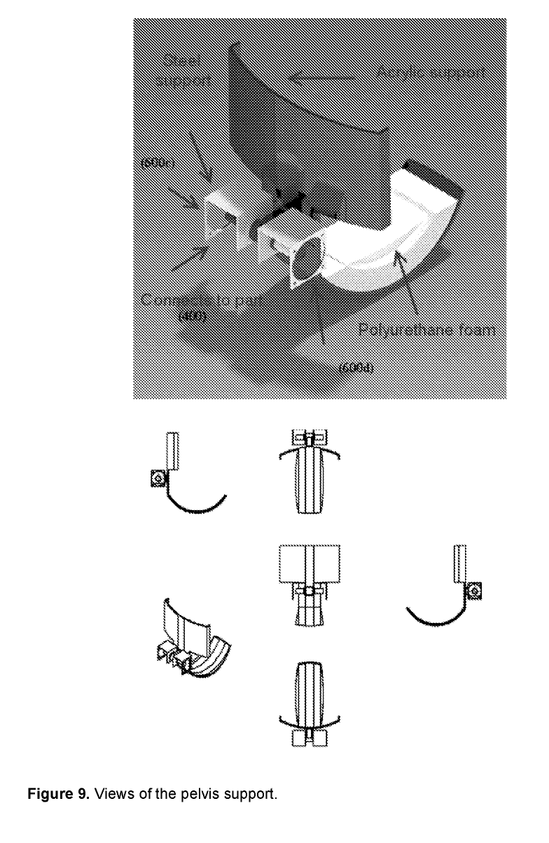

The pelvis support (100) in FIG. 9 mainly comprises a “U”-shaped seat (101), a back (102) and two connections (103). Said “U”-shaped seat (101) has a cylindrical shaft (104) at one of its extremities which is housed in hollow shaft (105) holding back (102) at the base.

Connections (103) form two parallel square covers joined by a square cover (103c) located perpendicularly to said covers (103a and 103b) and to the extremities of the covers (103a and 103b). Said cover (103a) has a hollow shaft which allows cylindrical shaft (204) to be inserted and in this way connections (103) connect with seat (101) and back (102) located at the rear of said back (102). Connections (103) are connected to seat (101) and back (102) in such a way that the outer part of cover (103b) for each of the connections is facing the same direction, and in such a way that the covers (103b) of the connectors face away from pelvis support (100). Cover (103b) of the connection has a hollow shaft with spaces for bolts.

Thus these covers (103b) have structures for mounting the bodies of the motors (600c and 600d), which allow the lower shoulder to flex and extend and the pelvis to rotate about the connecting shaft.

The hollow shafts of the covers (103b) of connections (103) make it possible for pelvis support (100) to be connected to upper leg brace (500) and chest support (100).

In this way upper leg brace (500) is joined to pelvis support (100) through attachment of the assembly formed by upper hollow shaft (503), motor (600c) and bearing (106) to the left hand connection (103) of said pelvis support (100). Chest support (200) is attached to pelvis support (100) through attachment of the assembly comprising lower hollow shaft (202), chest support (200), motor (600d) and bearing (107) to the right hand connection (103) of said pelvis support (100).

Unlike the components described above the pelvis support is preferably made of plastics because of its complex shape, which together with foam coverings on the lower and rear parts are intended to provide comfort for the user.

Chest Support (200):

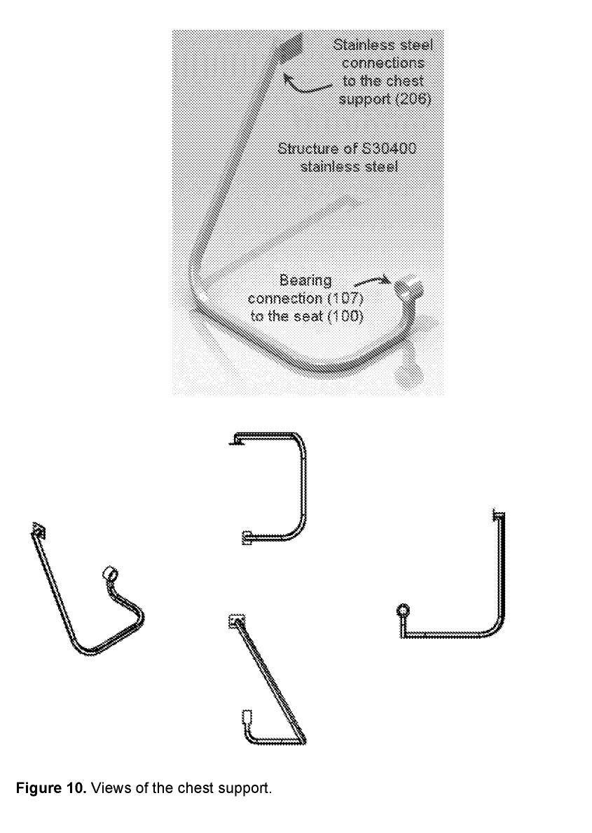

This component illustrated in FIG. 10 is the one responsible for reducing the loads that the upper part of the user's trunk produces on the lower shoulder. Said chest support (200) is designed to provide support for the chest in such a way that it does not inconvenience the surgeon when using device (1) and at the same time enables him to have the freedom of movement which he requires in the upper limbs.

In the same way as for the leg braces (400 and 500), this component comprises a square structural member (201) and shafts (202 and 203) machined as needed at the extremities of said component (200). Structural member (201) has a special shape which goes around the user's pelvis and rises at an angle of approximately 60° for the trunk as far as the chest (a distance which can vary between 500 mm and 600 mm) without limiting or obstructing possible movements of the upper limbs, as illustrated in FIG. 10.

Lower shaft (202) is a hollow shaft which allows radial ball race (107) to be connected in such a way that it joins this component (200) to the shaft of the right hand connection (103) of pelvis support (100) and at the same time provides a connection with motor shaft (600d) so that in this way there can be rotational freedom of movement between the user's shoulder and pelvis around the axis of chest support (200). At the upper extremity of chest support (200) there is a plate (204) to which a mounting (205) which is in turn attached to a chest support (206) can be attached.

Control System.

The device according to the invention also comprises a system (800) for controlling the position of the components of the structure of device (1). This control system (800) comprises a panel (801), a PLC controller (802), motors (600) and the coding units (601) for each motor.

Panel (801) is located outside the orthosis so that it can be operated by an assistant in accordance with the surgeon's instructions, because for sterility reasons the latter cannot touch it. This panel makes it possible to change programmed positions depending upon the user's needs, and for this the user can choose between 1 and 5 different positions through operating panel (801). The positions usually used by surgeons during surgery normally have an inclination of between 0 and 20° between the spine and the vertical. For this reason the device according to the invention is designed to adopt between 1 and 5 separate positions offering this range of inclination for the user's spine.

Panel (801) is preferably a touch control panel with SGC protocol, in which the various positions which the user will adopt are programmed. When the user selects a position the panel communicates with a PLC controller via SGC protocol, and the controller is programmed to communicate with the panel through serial transmission and reception and analyze the signals obtained, driving motors (600a, 600b, 600c and 600d) until they reach the desired positions, which can be measured using the coding units which are incorporated in the said motors. On reaching the desired position the locking system is activated by activating the solenoid so that it is finally fixed in the desired position supporting the surgeon's body loads.

1. Active orthosis device which can be adjusted to programmed positions for use in operating theaters to reduce the loads on surgeon's joints during long-term surgery,
wherein the said device comprises a mechanical structure with the ability to be locked, an electronic control system and a panel which can be used to control positions in accordance with the surgeon's posture and needs;
in which the mechanical structure mainly comprises a pelvis support; a chest support; a base; a lower leg brace; an upper leg brace; in which the lower leg brace is attached to the base through articulation; the lower leg brace and the upper leg brace are connected through articulation; the upper leg brace and the pelvis support are connected through articulation; and the upper leg brace and the chest support are connected through articulation;
four identical motors installed in the articulations of the device;
a system of springs which reduces the loads which have to be transmitted by the motors;
a locking system in the lower articulation; in which the said locking system comprises a solenoid and a pin guide provided in the base.

2. Active orthosis device according to claim 1 wherein the base is a flat sheet comprising
holes for anchoring to the floor by means of bolts; foot supports; a main support which houses the motor; a supporting shaft for the lower articulation to join the lower leg brace to the base;
in which the shaft supporting the lower articulation has a pierced circular support which allows the shaft of the solenoid to be inserted in the different positions programmed for the device; and a mount within which a torsion spring is located.

3. Active orthosis device according to claim 1, wherein the lower leg brace comprises a square structural component and two shafts each located at the extremities of the structural component; the shaft is located at the lower extremity; the said shaft houses a radial ball race mounted on the supporting shaft for the lower articulation of the base and the shaft of the lower motor;
in which the said hollow shaft located in the upper extremity houses the motor;
the said lower leg brace also comprises a housing support which attaches the solenoid of the locking system to the structure of the lower leg brace.

4. Active orthosis device according to claim 1, wherein the upper leg brace comprises a square structural element with an angle of between 30° and 35°, and two machined shafts at the extremities;
the said machined shaft at the lower extremity is attached to the motor and has two shafts of different diameter, the larger diameter fitting within the bearing; in which the smaller shaft diameter of the upper leg brace is attached to the motor.

5. Active orthosis device according to claim 1, wherein the pelvis support mainly comprises a “U”-shaped seat, a back and two connections;
in which the said seat has at one of its extremities a cylindrical shaft housed in the hollow shaft which holds the back at the base thereof;
in which the connections take the form of two parallel square covers joined by a square cover located perpendicularly to the said covers and the extremities of the covers; the said cover having a hollow shaft which allows the cylindrical shaft to be housed within;
in which the connections are attached to the seat and the back at the rear of the said back, and in which the exterior part of the cover of each of the connections is located in the same direction away from the pelvis support; in which the cover of the connection has a hollow shaft with holes for bolts;
the said covers comprise structures for mounting the bodies of the motors;
in which the upper leg support is attached to the pelvis support by means of the assembly of the upper hollow shaft, the motor, a bearing and the hollow shaft of the cover of the left-hand connection.

6. Active orthosis device according to claim 1, wherein the chest support comprises a square structural member and machined shafts, the said structural member having a length of between 500 mm and 600 mm and has a shape which surrounds the pelvis of the user of the device and rises at an angle of approximately 60° along the user's trunk as far as the chest;
the said lower shaft is a hollow shaft connecting a radial ball race and is connected to the shaft of the right-hand connector of the pelvis support and to the shaft of the motor;
the said upper extremity of the chest support has a plate which is attached to a mount which is in turn attached to a chest support;
in which the chest support is attached to the pelvis support through the assembly of the lower hollow shaft, the motor, a bearing and the hollow shaft of the cover of the right-hand connector.

7. Active orthosis device according to claim 1, wherein the control system comprises a touch screen, a PLC controller and coding units for each of the said motors;
the said touch screen is located outside the device and makes it possible for between 1 to 5 different positions of the components of the device to be selected.

8. Active orthosis device according to claim 7, wherein in the programmed positions the user's spine has an inclination of between 0 and 20 degrees with respect to the vertical.

9. Active orthosis device according to claim 7, wherein the coding units have a resolution of between 2000 pulses and 2500 pulses per revolution.

10. Active orthosis device according to claim 1, wherein the motors produce a torque of between 11 Nm and 18 Nm and an angular velocity of between 60 rad/min and 70 rad/min.
